(19) United States
(12) Patent Application Publication
(10) Pub. No.: US 2025/0239341 A1
(43) Pub. Date: Jul. 24, 2025

(54) Medical Paperwork Delivery System

(71) Applicant: Lori Misiaszek, Oriskany Falls, NY (US)

(72) Inventor: Lori Misiaszek, Oriskany Falls, NY (US)

(21) Appl. No.: 18/973,252

(22) Filed: Dec. 9, 2024

Related U.S. Application Data
(60) Provisional application No. 63/622,252, filed on Jan. 18, 2024.

(51) Int. Cl. G16H 15/00; G16H 10/40; G16H 10/60; G16H 20/10

(52) U.S. Cl. CPC G16H 15/00; G16H 10/40; G16H 10/60; G16H 20/10

(57) ABSTRACT

A medical paperwork delivery system is provided. The system is designed to facilitate the digitalization and transmission of medical paperwork, such as lab work, x-rays, and physical therapy orders. The system comprises at least one digital scanning device, which can be any device capable of converting paper documents into digital form, including but not limited to mobile devices with scanning applications and various types of scanners. After scanning the documents, a communication interface allows a user to select and send the digital document to another user, such as a medical lab or building. Additionally, the system enables a third party, like a patient, to access the document either through a mobile application or via other delivery means like text messages or emails. This accessibility allows the document to be printed or accessed digitally, facilitating a streamlined process for managing medical paperwork.

### CROSS-REFERENCE TO RELATED APPLICATION

The present application claims priority to, and the benefit of, U.S. Provisional Application No. 63/622,252, which was filed on Jan. 18, 2024, and is incorporated herein by reference in its entirety.

### FIELD OF THE INVENTION

The present invention relates generally to the field of medical paperwork. More specifically, the present invention relates to a medical paperwork delivery system that enables the conversion of medical paperwork into digital form using a digital scanning device and facilitates the sharing of these documents through a communication interface. Accordingly, the present disclosure makes specific reference thereto. Nonetheless, it is to be appreciated that aspects of the present invention are also equally applicable to other like applications, devices, and methods of manufacture.

### BACKGROUND

Medical patients who find themselves in need of laboratory work, such as blood tests or diagnostic screenings, often face a cumbersome process that can lead to unnecessary visits to emergency rooms or urgent care centers. This situation arises when individuals could have originally obtained these services through their primary care physicians. Typically, doctors will issue a paper slip to the patient, which serves as a formal request or prescription for the required lab work. This slip is an essential document, as it details the specific tests to be conducted and carries the doctor's authorization.

However, the reliance on physical paper slips poses several challenges. Patients may misplace or lose these slips, rendering them unable to proceed with the lab work until they acquire a replacement. The process of obtaining a new slip can be inconvenient and time-consuming. Patients must either visit their doctor's office in person to pick up another slip or wait for it to be mailed to them. This delay not only hampers the efficiency of healthcare delivery but also can lead to significant anxiety for the patient, who may be awaiting important test results.

In cases where the original slip is lost and immediate access to lab services is needed, patients are compelled to seek alternatives. Often, this means going to an emergency room or urgent care facility to request the lab work that could have been completed with the initial slip from their primary care doctor. Such detours not only place additional strain on emergency and urgent care resources, which are meant for more acute healthcare needs but also introduce potential delays and added costs for the patient. This inefficiency underscores a broader issue within healthcare systems regarding the reliance on paper-based processes and the need for more streamlined, digital solutions to facilitate patient care and lab work authorization.

Therefore, there exists a long-felt need in the art for an improved method and system for ordering medical tests and handling medical paperwork. There also exists a long-felt need in the art for a medical paperwork delivery system. More specifically, there exists a long-felt need in the art for a medical paperwork delivery system that minimizes the risk of losing a medical script and provides improved patient convenience. Further, there exists a long-felt need in the art for a medical paperwork delivery system that allows for the digital sending of medical scripts and other similar medical paperwork.

The subject matter disclosed and claimed herein, in one embodiment thereof, comprises a medical paperwork delivery system. The system is designed to facilitate the digitalization and transmission of medical paperwork, such as lab work, x-rays, and physical therapy orders. The system comprises at least one digital scanning device, which can be any device capable of converting paper documents into digital form, including but not limited to mobile devices with scanning applications and various types of scanners. After scanning the documents, a communication interface allows a user to select and send the digital document to another user, such as a medical lab or building. Additionally, the system enables a third party, like a patient, to access the document either through a mobile application or via other delivery means like text messages or emails. This accessibility allows the document to be printed or accessed digitally, facilitating a streamlined process for managing medical paperwork.

In this manner, the medical paperwork delivery system of the present invention accomplishes all the forgoing objectives and provides an improved method and system for ordering medical tests and handling medical paperwork. More specifically, the system reduces the risk of losing a medical script and provides improved patient convenience by digitizing the script ordering process. As a result, the medical script provider-to-patient interaction is greatly improved.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a medical paperwork delivery system that allows a first user (such as but not limited to a doctor or other medical provider) to order lab work, x-rays, physical therapy orders, and other similar medical paperwork of the like. To do so, the system is comprised of at least one digital scanning device that allows a user to scan physical lab orders (or other similar medical paperwork of the like) into digital format. Once scanned into digital format, the system provides a communication interface that allows the user to select the scanned document using the interface and virtually send the document to a second user, such as but not limited to a medical lab, a medical building, etc. such that said user is in possession of the document (ex. a script).

A third user, such as but not limited to a patient, can also access the document via a mobile application. In another embodiment, the document may be delivered to the third user via a delivery means such as but not limited to a text message, an email, etc. The application and/or delivery means then allows a user to print the document and/or access a link to a digital version of the document.

The present invention is also comprised of a method of using the device. First, a system is provided comprised of at least one scanner. Then, a first user (such as but not limited to a medical provider) can scan a physical script document (or other similar medical paperwork of the like) into a digital format using the scanner. Then, the first user can send the digital document to a lab or other similar medical facility via a communication interface of the system. A patient can then view the document via a mobile application or other delivery means and print the document as needed.

The present invention is also comprised of a second method of using the device. First, a system is provided comprised of at least one scanner. Then, a first user (such as but not limited to a medical provider) can scan a physical script document (or other similar medical paperwork of the like) into a digital format using the scanner. Then, the first user can send the digital document to a lab or other similar medical facility via a communication interface of the system. A patient can then view the document via a link sent to their smart device and print the document as needed.

Accordingly, the medical paperwork delivery system of the present invention is particularly advantageous as it provides an improved method and system for ordering medical tests and handling medical paperwork. More specifically, the system reduces the risk of losing a medical script and provides improved patient convenience by digitizing the script ordering process. As a result, the medical script provider-to-patient interaction is greatly improved. In this manner, the medical paperwork delivery system overcomes the limitations of existing methods of ordering medical scripts known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a graphical view of components of one potential embodiment of a medical paperwork delivery system of the present invention in accordance with the disclosed architecture;

FIG. 2 illustrates a flowchart of a method of using one potential embodiment of a medical paperwork delivery system of the present invention in accordance with the disclosed architecture; and

FIG. 3 illustrates a flowchart of a method of using one potential embodiment of a medical paperwork delivery system of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved method and system for ordering medical tests and handling medical paperwork. There also exists a long-felt need in the art for a medical paperwork delivery system. More specifically, there exists a long-felt need in the art for a medical paperwork delivery system that minimizes the risk of losing a medical script and provides improved patient convenience. Further, there exists a long-felt need in the art for a medical paperwork delivery system that allows for the digital sending of medical scripts and other similar medical paperwork.

The present invention, in one exemplary embodiment, is comprised of a medical paperwork delivery system. The system allows a first user (such as but not limited to a doctor or other medical provider) to order lab work, x-rays, physical therapy orders, and other similar medical paperwork of the like. To do so, the system is comprised of at least one digital scanning device. The device allows a user to scan physical lab orders (or other similar medical paperwork of the like) into digital format. Once scanned into digital format, the system provides a communication interface that allows the user to select the scanned document using the interface and virtually send the document to a second user, such as but not limited to a medical lab, a medical building, etc. such that said user is in possession of the document (ex. a script).

A third user, such as but not limited to a patient, can also access the document via a mobile application. In another embodiment, the document may be delivered to the third user via a delivery means such as but not limited to a text message, an email, etc. The application and/or delivery means then allows a user to print the document and/or access a link to a digital version of the document.

The present invention is also comprised of a method of using the device. First, a system is provided comprised of at least one scanner. Then, a first user (such as but not limited to a medical provider) can scan a physical script document (or other similar medical paperwork of the like) into a digital format using the scanner. Then, the first user can send the digital document to a lab or other similar medical facility via a communication interface of the system. A patient can then view the document via a mobile application or other delivery means and print the document as needed.

The present invention is also comprised of a second method of using the device. First, a system is provided comprised of at least one scanner. Then, a first user (such as but not limited to a medical provider) can scan a physical script document (or other similar medical paperwork of the like) into a digital format using the scanner. Then, the first user can send the digital document to a lab or other similar medical facility via a communication interface of the system. A patient can then view the document via a link sent to their smart device and print the document as needed.

Accordingly, the medical paperwork delivery system of the present invention is particularly advantageous as it provides an improved method and system for ordering medical tests and handling medical paperwork. More specifically, the system reduces the risk of losing a medical script and provides improved patient convenience by digitizing the script ordering process. As a result, the medical script provider-to-patient interaction is greatly improved. In this manner, the medical paperwork delivery system overcomes the limitations of existing methods of ordering medical scripts known in the art.

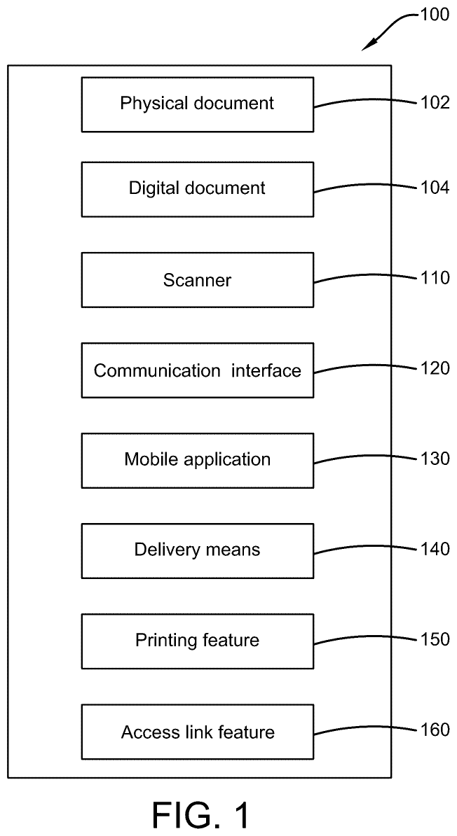

Referring initially to the drawings, FIG. 1 illustrates a graphical view of components of one potential embodiment of a medical paperwork delivery system 100 of the present invention in accordance with the disclosed architecture. The system 100 allows a first user (such as but not limited to a doctor or other medical provider) to order lab work, x-rays, physical therapy orders, and other similar medical paperwork of the like. To do so, the system 100 is comprised of at least one digital scanning device 110. The scanning device 110 may be any scanning device that allows a user to scan a physical document 102 into a digital document 104 such as but not limited to a mobile device with a scanning application, a flatbed scanner, a sheet-fed scanner, a portable scanner, a document 102 scanner, a photo scanner, a drum scanner, a handheld scanner, etc.

The scanning device 100 allows a first user to scan physical document 102 lab orders (or other similar medical paperwork of the like) into a digital document 104. Once scanned into digital format, the system 100 provides a communication interface 120. More specifically, the communication interface 120 may be present as interactive software on the scanner 110 or may be comprised of a mobile application or a computer software that can be accessed on any smart device such as but not limited to a smartphone, a computer, etc. As a result, a user can select the digital document 104 using the interface 120 and virtually send the document 104 to a second user, such as but not limited to a medical lab, a medical building, etc. such that said user is in possession of the document 104 (ex. a medical script, a lab order, etc.).

A third user, such as but not limited to a patient, can also access the document 104. In one embodiment, the document 104 can be accessed via a mobile application 130. In another embodiment, the document 104 may be delivered to the third user via a delivery means 140 such as but not limited to a text message, an email, etc. The application 130 and/or delivery means 140 then allows a user to print 150 the document 104 back into a physical document 102 and/or access a link 160 to a digital version of the document 104.

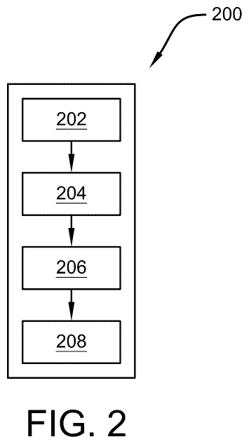

The present invention is also comprised of a method of using 200 the device 100, as seen in FIG. 2. First, a system 100 is provided comprised of at least one scanner 110 [Step 202]. Then, a first user (such as but not limited to a medical provider) can scan a physical script document 102 (or other similar medical paperwork of the like) into a digital document 104 using the scanner 110 [Step 204]. Then, the first user can send the digital document 104 to a lab or other similar medical facility via a communication interface 120 of the system 100 [Step 206]. A patient can then view the document 104 via a mobile application 130 or other delivery means 140 and print the document 104 as needed [Step 208].

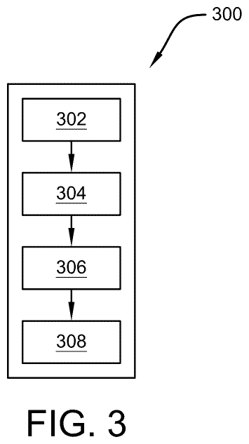

The present invention is also comprised of a second method of using 300 the device 100, as seen in FIG. 3. First, a system 100 is provided comprised of at least one scanner 110 [Step 302]. Then, a first user (such as but not limited to a medical provider) can scan a physical script document 102 (or other similar medical paperwork of the like) into a digital document 104 using the scanner 110 [Step 304]. Then, the first user can send the digital document 104 to a lab or other similar medical facility via a communication interface 120 of the system 100 [Step 306]. A patient can then view the document 104 via a link 160 sent to their smart device and print the document 104 as needed [Step 308].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “medical paperwork delivery system” and “system” are interchangeable and refer to the medical paperwork delivery system 100 of the present invention.

Notwithstanding the forgoing, the medical paperwork delivery system 100 of the present invention and its various components can be of any suitable size and configuration as is known in the art without affecting the overall concept of the invention, provided that they accomplish the above-stated objectives. One of ordinary skill in the art will appreciate that the size, configuration, and material of the medical paperwork delivery system 100 as shown in the FIGS. are for illustrative purposes only, and that many other sizes and shapes of the medical paperwork delivery system 100 are well within the scope of the present disclosure. Although the dimensions of the medical paperwork delivery system 100 are important design parameters for user convenience, the medical paperwork delivery system 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A medical paperwork delivery system comprising:
a document;
a scanner;
a communication interface;
a mobile application; and
a printing feature.

2. The medical paperwork delivery system of claim 1, wherein the document is comprised of a medical script or a lab order.

3. The medical paperwork delivery system of claim 1, wherein the scanner is comprised of a mobile device with a scanning application, a flatbed scanner, a sheet-fed scanner, a portable scanner, a document scanner, a photo scanner, a drum scanner, or a handheld scanner.

4. The medical paperwork delivery system of claim 1, wherein the scanner digitizes the document.

5. The medical paperwork delivery system of claim 1, wherein the communication interface is comprised of a software of the scanner.

6. The medical paperwork delivery system of claim 4, wherein the communication interface allows for transmission of the document once the document is converted to a digital scan via the scanner.

7. The medical paperwork delivery system of claim 4, wherein the document can be viewed by the mobile application.

8. The medical paperwork delivery system of claim 1, wherein the printing features allows for the document to be printed from the mobile application.

9. A medical paperwork delivery system comprising:
a document;
a scanner;
a communication interface;
a delivery means; and
an access link feature.

10. The medical paperwork delivery system of claim 9, wherein the document is comprised of a medical script or a lab order.

11. The medical paperwork delivery system of claim 9, wherein the scanner is comprised of a mobile device with a scanning application, a flatbed scanner, a sheet-fed scanner, a portable scanner, a document scanner, a photo scanner, a drum scanner, or a handheld scanner.

12. The medical paperwork delivery system of claim 9, wherein the scanner digitizes the document.

13. The medical paperwork delivery system of claim 9, wherein the communication interface is comprised of a software of the scanner.

14. The medical paperwork delivery system of claim 9, wherein the communication interface allows for transmission of the document once the document is converted to a digital scan via the scanner.

15. The medical paperwork delivery system of claim 12, wherein the access link feature provides a user with access to a digital version of the document.

16. A method of using a medical paperwork delivery system, the method comprising the following steps:
providing a medical paperwork delivery system comprised of a scanner;
scanning a physical script document into a digital document using the scanner;
sending the digital document to a medical facility via a communication interface of the medical paperwork delivery system; and
viewing the digital document via a mobile application of the medical paperwork delivery system or an access link feature of the medical paperwork delivery system.

17. The method of using a medical paperwork delivery system of claim 16, wherein the scanner is comprised of a mobile device with a scanning application, a flatbed scanner, a sheet-fed scanner, a portable scanner, a document scanner, a photo scanner, a drum scanner, or a handheld scanner.

18. The method of using a medical paperwork delivery system of claim 16, wherein the communication interface is comprised of a software of the scanner.

19. The method of using a medical paperwork delivery system of claim 16, wherein the access link feature provides a user with access to the digital document.

20. The method of using a medical paperwork delivery system of claim 16, wherein the mobile application provides a user with access to the digital document.
